(19) United States
(12) Patent Application Publication
(10) Pub. No.: US 2024/0428913 A1
(43) Pub. Date: Dec. 26, 2024

(54) PSILOCYBIN THERAPY FOR TREATMENT RESISTANT DEPRESSION

(71) Applicant: Compass Pathfinder Limited, Altrincham (GB)

(72) Inventors: Robert F. Dougherty, Redwood City, CA (US); Patrick Clarke, Philadelpia, PA (US); Gregory A. Ryslik, Westerville, OH (US)

(21) Appl. No.: 18/274,066

(22) PCT Filed: Jul. 24, 2023
(86) PCT No.: PCT/US23/70857

§ 371 Date: Jul. 25, 2023

Related U.S. Application Data
(60) Provisional application No. 63/414,769, filed on Oct. 10, 2022.
(60) Provisional application No. 63/392,451, filed on Jul. 26, 2022.

(51) Int. Cl. G16H 20/10; G16H 50/70

(52) U.S. Cl. CPC G16H 20/10; G16H 50/70

(57) ABSTRACT

Approaches for predicting a response to an administered therapy are provided. One or more recordings of a session related to an administered therapy may be transcribed into one or more transcripts. The transcripts may be parsed into utterances. An utterance sentiment may be determined for individual utterances, and a response to the administered therapy may be predicted based, at least in part, upon the utterance sentiment.

### CROSS-REFERENCE TO RELATED APPLICATIONS

This application claims priority to and the benefit of PCT Application Serial No. PCT/US23/70857 filed Jul. 24, 2023 titled “PSILOCYBIN THERAPY FOR TREATMENT RESISTANT DEPRESSION,” and U.S. Provisional Application Ser. No. 63/392,451 filed Jul. 26, 2022 titled “PSILOCYBIN THERAPY FOR TREATMENT RESISTANT DEPRESSION,” and U.S. Provisional Application Ser. No. 63/414,769 filed Oct. 10, 2022 titled “PSILOCYBIN THERAPY FOR TREATMENT RESISTANT DEPRESSION,” the full disclosures of which are hereby incorporated herein by reference in their entirety for all purposes.

### BACKGROUND

Therapeutic administration of psychedelic drugs has shown significant potential, both in historical accounts and in recent clinical trials on the treatment of depression and other mood disorders. For example, recent studies have shown promising results when using psilocybin formulations for patients with treatment-resistant depression (TRD). However, while promising, such a treatment may only work for a portion of the population, and early prediction of an outcome is a key objective for treatment.

### BRIEF DESCRIPTION OF THE DRAWINGS

Various embodiments in accordance with the present disclosure will be described with reference to the drawings, in which:

FIG. 1 illustrates an example sentiment plot that can be utilized in accordance with various embodiments.

FIG. 2 illustrates an example method that can be utilized to implement one or more aspects of the various embodiments.

FIG. 3 illustrates an example method that can be utilized to implement one or more aspects of the various embodiments.

FIG. 4 illustrates components of an example computing device that can be utilized in accordance with various embodiments.

FIG. 5 illustrates an example of an environment for implementing aspects in accordance with various embodiments.

FIG. 6 illustrates components of another example environment in which aspects of various embodiments can be implemented.

### DETAILED DESCRIPTION

Various factors may be considered when predicting a patient's potential response to a therapy. For example, in combining average linguistic sentiment scores of a patient and therapist during a post-dosing psychological support session or integration session a machine learning model may be fit to predict a therapeutic response for participants at both upcoming and future time points with high fidelity. These approaches may be utilized in accordance with one or more embodiments to help predict at least part of a participant's or patient's response to psychedelic therapy.

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Systems and methods for predicting a response to an administered therapy are provided. One or more scores corresponding to an individual's reported results related to an administered therapy may be received. One or more recordings of a session related to the administered therapy may be transcribed into one or more transcripts. The transcripts may be parsed into utterances. An utterance sentiment may be determined for individual utterances, and a response to the administered therapy may be predicted based, at least in part, upon the scores and the utterance sentiment.

Major depressive disorder (MDD) is a debilitating disease and can affect one in six adults in their lifetime. MDD may be characterized by at least one depressive episode having a duration of at least two weeks and involving clear changes in mood, cognition, and the ability to experience pleasure. While MDD may be effectively managed using psychotherapy and/or pharmacological treatments, some MDD patients may not respond to treatment, despite multiple treatment attempts. Such individuals may be referred to as patients having treatment-resistant depression (TRD). There are various existing options for treatment, but they are often determined to be unsatisfactory. Therefore, there is a need for the development of alternative therapeutic options for TRD patients that have improved efficacy. Additionally, acceptability of a condition or treatment may remain an important challenge for such patients.

Psilocybin is a tryptamine alkaloid whose potential as an effective antidepressant was preliminarily studied in patients with life-threatening cancer, MDD, and TRD. In at least one psilocybin study using a 25 mg dose of COMP360, a set of participants experienced reduced depression symptoms for as many as 12 weeks. While very promising, these results show that a durable psilocybin response occurs in only a portion of the TRD population. Weeks may pass while potential opportunities for additional treatment are potentially wasted.

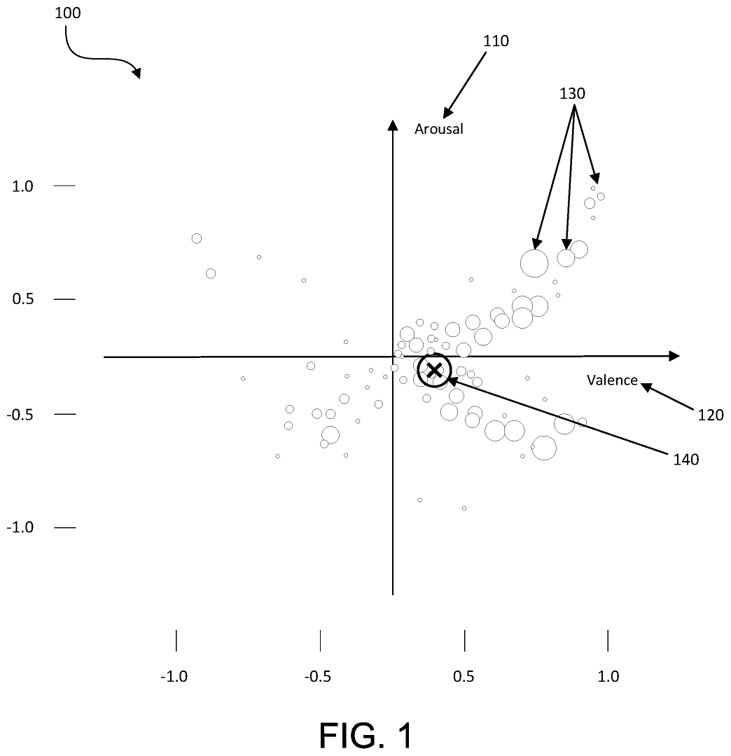

FIG. 1 illustrates an example sentiment plot 100 that can be utilized in accordance with various embodiments. In at least some embodiments, a model may be utilized to help predict whether a participant will respond at a particular week in their treatment timeline. The model may consider various factors, including the patient's and therapist's sentiment score, the participant or patient's treatment arm (dosage), and a measure of the average linguistic sentiment of the participant or patient and therapist during an integration session at least one day after dosing.

In some embodiments, a predictive model may be utilized to predict which participants would be responders, sustained responders, and relaxed sustained responders using information available immediately after treatment. A machine learning model may be utilized to help predict responses in patients. In an example embodiment, the machine learning model may account for additional exogenous variables, including, but not limited to, values summarizing participant or patient sentiment during an integration session, and values summarizing a therapist's sentiment during the integration session.

At least one day after initial psilocybin dosing, a patient or participant may have an integration session with a therapist to discuss their initial reactions to the treatment. Audio recordings of the integration session may be collected and transcribed into dialogue text, either manually or through the use of natural language processing (NLP) techniques. The transcripts may then be parsed into individual “utterances” used to estimate session sentiment for the therapist and the patient or participant using a sentiment model. Such a model may produce valence and arousal scores for each utterance. As shown in FIG. 1, the arousal score 110 and valence score 120 may be represented on a sentiment plot 100. Individual circles, such as circles 130, may represent an utterance from the session, and a circle's size may reflect a relative length of the utterance. A circled “X”, such as element 140, may mark a mean sentiment value of all utterances during a session. The sentiment plot may be patient or therapist specific. The valence score may correspond to a positive or negative score used in sentiment analysis. An arousal score may characterize where the utterance lies on the spectrum, such as bored or calm to tense, alert, or excited. Scores may indicate an average sentiment of the utterances spoken by a patient or participant and therapist during the integration session.

In at least some embodiments, audio may be parsed and tagged to the utterances. The audio may be processed using a model to predict a patient's tone. In some embodiments, the audio may be correlated with a score indicative of a patient's tone. The audio score may be compared to the valence and arousal scores, or the audio score may be accounted for with the valence and arousal scores. A classifier built on a Bidirectional AutoRegressive Transformer (BART) autoencoder and Multi-Genre Natural Language Inference (MNLI) dataset may be used to calculate the sentiment valence and arousal scores.

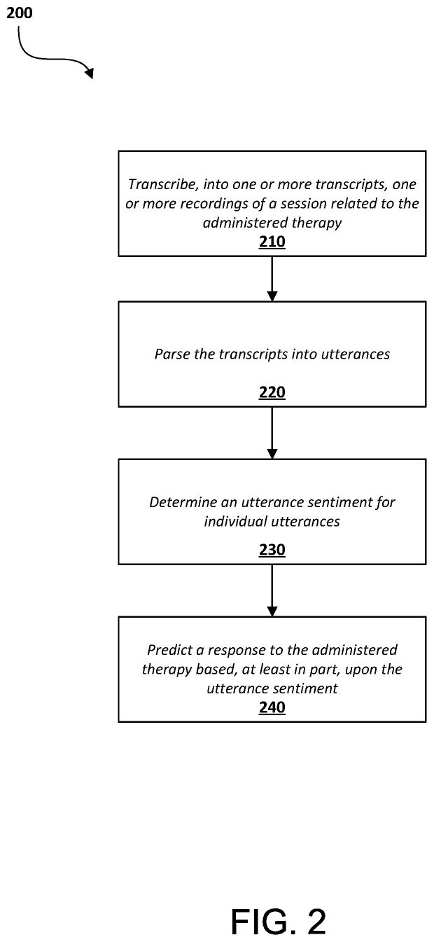

FIG. 2 illustrates an example method 200 that can be utilized to implement one or more aspects of the various embodiments. An audio recording may be generated at the time of the initial integration session between patient or participant and the medical provider, such as a therapist. Using one or more NLP techniques, the recording may be transcribed to text 210, and the text may be parsed to generate a set of utterances 220. Sentiment scores may be assigned or determined for individual utterances of the set 230. Session averages of the sentiment scores may be computed. The averaged sentiment scores may be utilized to predict a patient's response to treatment.

Prior to a psychological support session with a therapist or medical care provider, the patient or participant may provide a written consent form to indicate consent to be recorded during the session. Sessions may be recorded using any device comprising a microphone. In some embodiments, a recording device may be pre-configured with a secure cloud storage account. Once recordings are completed, they may be automatically uploaded to the configured storage account. Quality assurance checks may be performed to ensure the metadata for individual recordings are accurate, and recordings may be made available for transcription.

Audio recordings may be manually or automatically transcribed. Identifiable information of both the patient or participant and a healthcare provider may be removed to anonymize the transcripts. As mentioned, transcripts may be reviewed for quality assurance prior to analysis. After converting the audio recording to text, individual transcripts may be parsed into utterances. In some embodiments, transcript punctuation may be utilized as a guide. A trained large language model may be utilized, in accordance with one or more embodiments, to help score utterances. For example, the model may analyze individual sentences that have been parsed and constructed with text that may be recognizable by the model to produce scores.

In NLP, an “utterance” may be defined as a spoken group of words that is preceded by and followed by a pause. In contrast, a “sentence” may refer to a group of words that express a complete thought. According to one or more embodiments described herein, an “utterance” may be an amalgamation of the two. For example, if a sentence occurs over several utterances, the utterances may be combined to form a single utterance. Alternatively, if an utterance contains several sentences, each sentence may be extracted and can be treated as stand-alone utterances.

Additional processing steps may be taken after transcribing. For example, Unicode characters may be converted to ASCII characters. Redacted information such as names may be replaced with non-personally identifiable alternatives (e.g., Jane/John Doe). According to another example embodiment, transcriber comments may be replaced with model-familiar text. For example, a transcription note “[LAUGHING]” may be replaced with “haha” so that the model may better-recognize the term and use the term in generating scores.

Sentiment analysis of a piece of text may include scoring the text as being positive or negative. Text may be scored in two dimensions: valence (e.g., positivity) and arousal (e.g., energy or activation). In this way, sentiment may capture intensity, rather than just positivity or negativity. The sentiment score of a piece of text may be distinguishable from trying to infer an emotional state of a speaker. For example, text reciting “I love broccoli” may be scored by traditional sentiment models as being positive. However, if vocalized in a sarcastic way, the text would signal a negative attitude towards broccoli. Additionally, text reciting “I love broccoli” may be ranked as 97% positive in one model, and text reciting “I like broccoli” may be ranked as 98% positive in the same model. The similarity in positivity score may be a result of the sentiment analysis problem being treated as a classification problem, and not considering intensity. In such cases, the model may only care about whether the “positive” or “negative” label is correct.

A two-dimensional emotional model may plot an emotion in the xy-plane, where the x-value represents valence (e.g., positivity) and the y-value represents arousal (e.g., energy or activation). Examples of high valence emotions, according to an example embodiment, may include “giddy,” “happy,” “content,” and “serene.” Low valence emotions, according to an example embodiment, may include “fear,” “nervousness,” “sadness,” and “boredom.” High arousal emotions, according to an example embodiment, may include “tense,” “alarmed,” “astonished,” and “excited.” Low arousal emotions, according to an example embodiment, may include “bored,” “droopy,” “tired,” and “sleepy.”

In some example embodiments, a classifier may be utilized to compute sentiment valence and arousal scores. Such a classifier may be built on a BART autoencoder and Multi-Genre Natural Language Inference (MNLI) dataset. The use of a model built on top of larger models may enable the use of almost unlimited freely available data for smaller data tasks. BART is a deep learning model designed to reconstruct text that had been corrupted. It was trained on a large corpus of books and online content. Fine-tuning BART on the MNLI dataset may result in a classifier for any given classes. For example, a user can specify a list of classes and submit a piece of text to be classified. Text reciting “I love broccoli” may be submitted to be classified into the classes of “food” and “politics,” where the model may indicate a 99.8% probability that this text is about food and a 0.2% probability that this text is about politics, for example.

In accordance with one or more embodiments described herein, for individual utterances, the model may be used to score the likelihood that the utterance should belong to one of the following four classes: “happy,” “angry,” “gloomy,” and “calm.” While the present example utilizes the aforementioned classes, other classes indicative of sentiment may be utilized. For a given utterance, the model may provide scores for individual classes. The scores may be positive numbers that sum to one, in an example embodiment. Valence-arousal score pairs may be assigned to the classes so that the scores may be utilized to plot a point in the two-dimensional emotional plane.

For example, for a given set of classes (class 1, class 2, class 3, class 4), the model may determine scores for class 1 to be (1, 1), scores for class 2 to be (−1, 1), scores for class 3 to be (−1, −1) and scores for class 4 to be (1, −1). In some embodiments, classes 1 and 2 may be pairs in terms of sentiment (e.g., “happy” and “angry”), and classes 3 and 4 may be pairs (e.g., “gloomy” and “calm”). The point (valence (utterance), arousal (utterance)) for a given utterance u may then be defined as the convex combination of the points (1, 1), (−1, 1), (1, −1) and (1, 1):

$$( valence ( utterance ) , arousal ( utterance ) ) = {P}_{u} ( class 1 ) ( 1 , 1 ) + {P}_{u} ( class 2 ) ( - 1 , 1 ) + {P}_{u} ( class 3 ) ( - 1 , - 1 ) + {P}_{u} ( class 4 ) ( 1 , - 1 ) \qquad ( Eq . 1 )$$

### Equivalently,

$$valence ( u ) = {P}_{u} ( class 1 ) - {P}_{u} ( class 2 ) - {P}_{u} ( class 3 ) + {P}_{u} ( class 4 ) \qquad ( Eq . 2 )$$

$$and$$

$$arousal ( u ) = {P}_{u} ( class 1 ) + {P}_{u} ( class 2 ) - {P}_{u} ( class 3 ) - {P}_{u} ( class 4 ) \qquad ( Eq . 3 )$$

These equations may result in a point that lies in a two-dimensional square with corners at (±1, ±1). In an example embodiment, one or more points may be plotted in a two-dimensional square, such as that shown in FIG. 1.

One or more machine learning models may be utilized to help predict class membership probability, such as responder vs. non-responder, for each subject. In at least some embodiments, the predictions may be cross validated to check for accuracy of the models. Valence and arousal scores may be extracted using classes such as “positive,” “negative,” “aroused,” or “unaroused,” but these classes may not be effective for extraction because the classes may be too broad to accurately capture a patient's sentiment.

To determine a session sentiment score, a set of utterances of a given speaker (e.g., participant or patient, therapist, etc.) during a single visit (e.g., first preparation, first integration, etc.) may be given a probability measure, where the measure of a given utterance is proportional to the number of words in the utterance. From this, mean valence and arousal scores may be obtained.

A response to the administered therapy may be predicted based, at least in part, upon the EBI summary score and determined values 250. In some embodiments, machine learning models may be utilized to predict whether a participant or patient is a responder, a relaxed sustained responder, or a sustained responder to treatment or therapy. In some example embodiments, a treatment or therapy may refer to administration of psilocybin. A responder, in some example embodiments, may be defined as a patient whose primary outcome measures score three weeks post-therapy administration date was reduced by at least 50% relative to their baseline score. A sustained responder may be defined as a participant or patient who had at least a 50% reduction in their score at each determined time point (e.g., weeks three, six, nine, and twelve). A relaxed sustained responder may be defined as a participant or patient who has had at least a 50% reduction in their score at a determined time point (e.g., week three and twelve), and one of the intervening weeks (e.g., one of weeks six or nine). This definition of a “responder” is not intended to be limiting, and other definitions of a “responder” may be utilized.

The machine learning models may utilize the patient's or participant's EBI score, treatment dose, and sentiment metrics from a transcript of the first integration session the day following treatment. The machine learning models may be fit to a full data set to ensure that the selected models resulted in an adequate fit. Leave-one-out (LOO) cross-validation may be performed to assess the predictive power of the machine learning models. For example, the model may be fit to all participants or patients except one and the model may be utilized to predict the excluded participant's responder status. The model, in some example embodiments, may be trained on existing data and utilized to predict the responder status of a new patient.

In some example embodiments, treatment may be encoded as categorical variables with values of 0, 1, and 2 for 1 mg, 10 mg, and 25 mg treatment groups, respectively. Additionally, therapy session sentiment scores may be normalized to have zero mean and a standard deviation of one.

It should be understood that for any process herein there can be additional, fewer, or alternative steps performed in similar or alternative orders, or in parallel, within the scope of the various embodiments unless otherwise specifically stated.

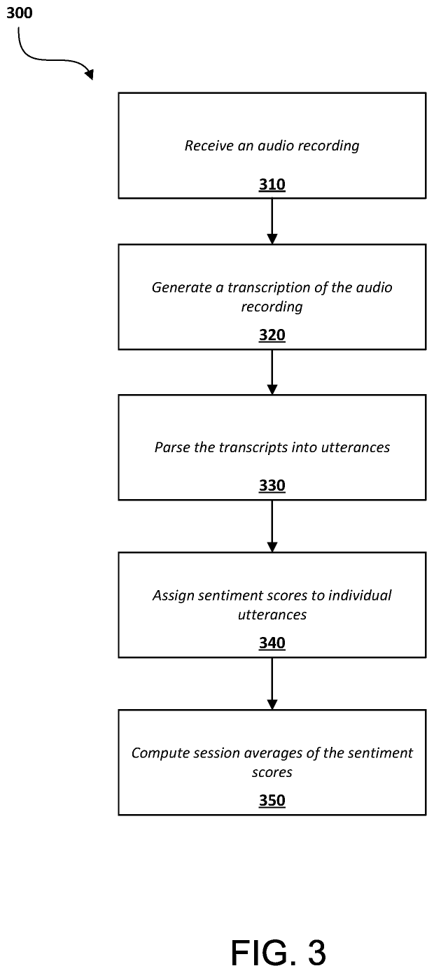

FIG. 3 illustrates an example method 300 that can be utilized to implement one or more aspects of the various embodiments. An audio recording of an initial integration session between the patient and medical provider may be generated and received 310. Using one or more NLP techniques, the recording may be transcribed to text 320, and the text may be parsed to generate a set of utterances 330. Sentiment scores may be assigned or determined for individual utterances of the set 340. Session averages of the sentiment scores may be computed 350. The averaged sentiment scores may be utilized to predict a patient's response to treatment.

Audio recordings may be manually or automatically transcribed. Identifiable information of both the patient or participant and a healthcare provider may be removed to anonymize the transcripts. As mentioned, transcripts may be reviewed for quality assurance prior to analysis. After converting the audio recording to text, individual transcripts may be parsed into utterances. In some embodiments, transcript punctuation may be utilized as a guide. A trained large language model may be utilized, in accordance with one or more embodiments, to help score utterances.

In NLP, an “utterance” may be defined as a spoken group of words that is preceded by and followed by a pause. In contrast, a “sentence” may refer to a group of words that express a complete thought. According to one or more embodiments described herein, an “utterance” may be an amalgamation of the two. For example, if a sentence occurs over several utterances, the utterances may be combined to form a single utterance. Alternatively, if an utterance contains several sentences, each sentence may be extracted and can be treated as stand-alone utterances.

Additional processing steps may be taken after transcribing. For example, Unicode characters may be converted to ASCII characters. Redacted information such as names may be replaced with non-personally identifiable alternatives (e.g., Jane/John Doe). According to another example embodiment, transcriber comments may be replaced with model-familiar text. For example, a transcription note “[LAUGHING]” may be replaced with “haha” so that the model may better-recognize the term and use the term in generating scores.

Sentiment analysis of a piece of text may include scoring the text as being positive or negative. Text may be scored in two dimensions: valence (e.g., positivity) and arousal (e.g., energy or activation). In this way, sentiment may capture intensity, rather than just positivity or negativity. Sentiment score of a piece of text may be distinguishable from trying to infer an emotional state of a speaker. For example, text reciting “I love broccoli” may be scored by traditional sentiment models as being positive. However, if vocalized in a sarcastic way, the text would signal a negative attitude towards broccoli. Additionally, text reciting “I love broccoli” may be ranked as 97% positive in one model, and text reciting “I like broccoli” may be ranked as 98% positive in the same model. The similarity in positivity score may be a result of the sentiment analysis problem being treated as a classification problem, and not considering intensity. In such cases, the model may only care about whether the “positive” or “negative” label is correct.

A two-dimensional emotional model may plot an emotion in the xy-plane, where the x-value represents valence (e.g., positivity) and the y-value represents arousal (e.g., energy or activation). Examples of high valence emotions, according to an example embodiment, may include “giddy,” “happy,” “content,” and “serene.” Low valence emotions, according to an example embodiment, may include “fear,” “nervousness,” “sadness,” and “boredom.” High arousal emotions, according to an example embodiment, may include “tense,” “alarmed,” “astonished,” and “excited.” Low arousal emotions, according to an example embodiment, may include “bored,” “droopy,” “tired,” and “sleepy.”

In some example embodiments, a classifier may be utilized to compute sentiment valence and arousal scores. Such a classifier may be built on a BART autoencoder and Multi-Genre Natural Language Inference (MNLI) dataset. The use of a model built on top of larger models may enable the use of almost unlimited freely available data for smaller data tasks. BART is a deep learning model designed to reconstruct text that had been corrupted. BART was trained on a large corpus of books and online content. Fine-tuning BART on the MNLI dataset may result in a classifier for any given classes. For example, a user can specify a list of classes and submit a piece of text to be classified. Text reciting “I love broccoli” may be submitted to be classified into the classes of “food” and “politics,” where the model may indicate a 99.8% probability that this text is about food and a 0.2% probability that this text is about politics, for example.

In accordance with one or more embodiments described herein, for individual utterances, the model may be used to score the likelihood that the utterance should belong to one of the following four classes: “happy,” “angry,” “gloomy,” and “calm.” While the present example utilizes the aforementioned classes, other classes indicative of sentiment may be utilized. For a given utterance, the model may provide scores for individual classes. The scores may be positive numbers that sum to one, in an example embodiment. Valence-arousal score pairs may be assigned to the classes so that the scores may be utilized to plot a point in the two-dimensional emotional plane.

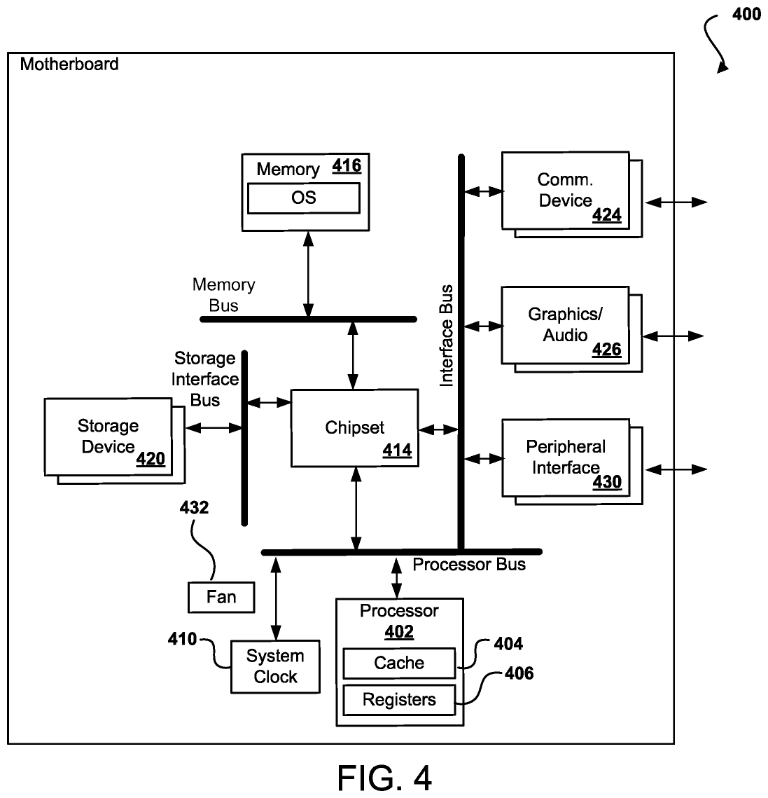

Computing resources, such as servers, that can have software and/or firmware updated in such a matter will generally include at least a set of standard components configured for general purpose operation, although various proprietary components and configurations can be used as well within the scope of the various embodiments. FIG. 4 illustrates components of an example computing device 400 that can be utilized in accordance with various embodiments. As known for computing devices, the computer will have one or more processors 402, such as central processing units (CPUs), graphics processing units (GPUs), and the like, that are electronically and/or communicatively coupled with various components using various buses, traces, and other such mechanisms. A processor 402 can include memory registers 406 and cache memory 404 for holding instructions, data, and the like. In this example, a chipset 414, which can include a northbridge and southbridge in some embodiments, can work with the various system buses to connect the processor 402 to components such as system memory 416, in the form or physical RAM or ROM, which can include the code for the operating system as well as various other instructions and data utilized for operation of the computing device. The computing device can also contain, or communicate with, one or more storage devices 420, such as hard drives, flash drives, optical storage, and the like, for persisting data and instructions similar, or in addition to, those stored in the processor and memory. The processor 402 can also communicate with various other components via the chipset 414 and an interface bus (or graphics bus, etc.), where those components can include communications devices 424 such as cellular modems or network cards, media components 426, such as graphics cards and audio components, and peripheral interfaces 430 for connecting peripheral devices, such as printers, keyboards, and the like. At least one cooling fan 432 or other such temperature regulating or reduction component can also be included as well, which can be driven by the processor or triggered by various other sensors or components on, or remote from, the device. Various other or alternative components and configurations can be utilized as well as known in the art for computing devices.

At least one processor 402 can obtain data from physical memory 416, such as a dynamic random-access memory (DRAM) module, via a coherency fabric in some embodiments. It should be understood that various architectures can be utilized for such a computing device, that may include varying selections, numbers, and arguments of buses and bridges within the scope of the various embodiments. The data in memory may be managed and accessed by a memory controller, such as a DDR controller, through the coherency fabric. The data may be temporarily stored in a processor cache 404 in at least some embodiments. The computing device 400 can also support multiple I/O devices using a set of I/O controllers connected via an I/O bus. There may be I/O controllers to support respective types of I/O devices, such as a universal serial bus (USB) device, data storage (e.g., flash or disk storage), a network card, a peripheral component interconnect express (PCIe) card or interface 430, a communication device 424, a graphics or audio card 426, and a direct memory access (DMA) card, among other such options. In some embodiments, components such as the processor, controllers, and caches can be configured on a single card, board, or chip (i.e., a system-on-chip implementation), while in other embodiments at least some of the components may be located in different locations, etc.

An operating system (OS) running on the processor 402 can help to manage the various devices that may be utilized to provide input to be processed. This can include, for example, utilizing relevant device drivers to enable interaction with various I/O devices, where those devices may relate to data storage, device communications, user interfaces, and the like. The various I/O devices will typically connect via various device ports and communicate with the processor and other device components over one or more buses. There can be specific types of buses that provide for communications according to specific protocols, as may include peripheral component interconnect) PCI or small computer system interface (SCSI) communications, among other such options. Communications can occur using registers associated with the respective ports, including registers such as data-in and data-out registers. Communications can also occur using memory mapped I/O, where a portion of the address space of a processor is mapped to a specific device, and data is written directly to, and from, that portion of the address space.

Such a device may be used, for example, as a server in a server farm or data warehouse. Server computers often have a need to perform tasks outside the environment of the CPU and main memory (i.e., RAM). For example, the server may need to communicate with external entities (e.g., other servers) or process data using an external processor (e.g., a General Purpose Graphical Processing Unit (GPGPU)). In such cases, the CPU may interface with one or more I/O devices. In some cases, these I/O devices may be special-purpose hardware designed to perform a specific role. For example, an Ethernet network interface controller (NIC) may be implemented as an application specific integrated circuit (ASIC) comprising digital logic operable to send and receive packets.

In an illustrative embodiment, a host computing device is associated with various hardware components, software components and respective configurations that facilitate the execution of I/O requests. One such component is an I/O adapter that inputs and/or outputs data along a communication channel. In one aspect, the I/O adapter device can communicate as a standard bridge component for facilitating access between various physical and emulated components and a communication channel. In another aspect, the I/O adapter device can include embedded microprocessors to allow the I/O adapter device to execute computer executable instructions related to the implementation of management functions or the management of one or more such management functions, or to execute other computer executable instructions related to the implementation of the I/O adapter device. In some embodiments, the I/O adapter device may be implemented using multiple discrete hardware elements, such as multiple cards or other devices. A management controller can be configured in such a way to be electrically isolated from any other component in the host device other than the I/O adapter device. In some embodiments, the I/O adapter device is attached externally to the host device. In some embodiments, the I/O adapter device is internally integrated into the host device. Also in communication with the I/O adapter device may be an external communication port component for establishing communication channels between the host device and one or more network-based services or other network-attached or direct-attached computing devices. Illustratively, the external communication port component can correspond to a network switch, sometimes known as a Top of Rack (“TOR”) switch. The I/O adapter device can utilize the external communication port component to maintain communication channels between one or more services and the host device, such as health check services, financial services, and the like.

The I/O adapter device can also be in communication with a Basic Input/Output System (BIOS) component. The BIOS component can include non-transitory executable code, often referred to as firmware, which can be executed by one or more processors and used to cause components of the host device to initialize and identify system devices such as the video display card, keyboard and mouse, hard disk drive, optical disc drive and other hardware. The BIOS component can also include or locate boot loader software that will be utilized to boot the host device. For example, in one embodiment, the BIOS component can include executable code that, when executed by a processor, causes the host device to attempt to locate Preboot Execution Environment (PXE) boot software. Additionally, the BIOS component can include or takes the benefit of a hardware latch that is electrically controlled by the I/O adapter device. The hardware latch can restrict access to one or more aspects of the BIOS component, such controlling modifications or configurations of the executable code maintained in the BIOS component. The BIOS component can be connected to (or in communication with) a number of additional computing device resources components, such as processors, memory, and the like. In one embodiment, such computing device resource components may be physical computing device resources in communication with other components via the communication channel. The communication channel can correspond to one or more communication buses, such as a shared bus (e.g., a processor bus, a memory bus), a point-to-point bus such as a PCI or PCI Express bus, etc., in which the components of the bare metal host device communicate. Other types of communication channels, communication media, communication buses or communication protocols (e.g., the Ethernet communication protocol) may also be utilized. Additionally, in other embodiments, one or more of the computing device resource components may be virtualized hardware components emulated by the host device. In such embodiments, the I/O adapter device can implement a management process in which a host device is configured with physical or emulated hardware components based on a variety of criteria. The computing device resource components may be in communication with the I/O adapter device via the communication channel. In addition, a communication channel may connect a PCI Express device to a CPU via a northbridge or host bridge, among other such options.

In communication with the I/O adapter device via the communication channel may be one or more controller components for managing hard drives or other forms of memory. An example of a controller component can be a SATA hard drive controller. Similar to the BIOS component, the controller components can include or take the benefit of a hardware latch that is electrically controlled by the I/O adapter device. The hardware latch can restrict access to one or more aspects of the controller component. Illustratively, the hardware latches may be controlled together or independently. For example, the I/O adapter device may selectively close a hardware latch for one or more components based on a trust level associated with a particular user. In another example, the I/O adapter device may selectively close a hardware latch for one or more components based on a trust level associated with an author or distributor of the executable code to be executed by the I/O adapter device. In a further example, the I/O adapter device may selectively close a hardware latch for one or more components based on a trust level associated with the component itself. The host device can also include additional components that are in communication with one or more of the illustrative components associated with the host device. Such components can include devices, such as one or more controllers in combination with one or more peripheral devices, such as hard disks or other storage devices. Additionally, the additional components of the host device can include another set of peripheral devices, such as Graphics Processing Units (“GPUs”). The peripheral devices and can also be associated with hardware latches for restricting access to one or more aspects of the component. As mentioned above, in one embodiment, the hardware latches may be controlled together or independently.

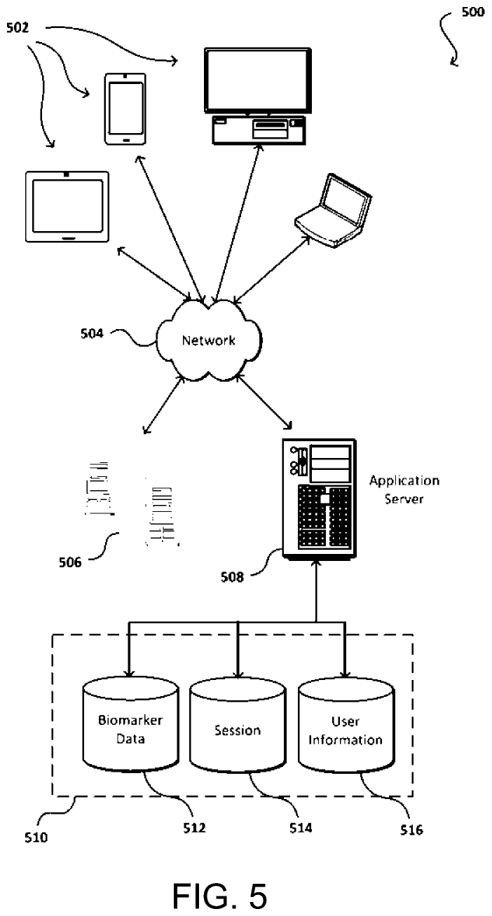

As discussed, different approaches can be implemented in various environments in accordance with the described embodiments. For example, FIG. 5 illustrates an example of an environment 500 for implementing aspects in accordance with various embodiments. As will be appreciated, although a Web-based environment is used for purposes of explanation, different environments may be used, as appropriate, to implement various embodiments. The system includes an electronic client device 502, which can include any appropriate device operable to send and receive requests, messages or information over an appropriate network 504 and convey information back to a user of the device. Examples of such client devices include personal computers, cell phones, handheld messaging devices, laptop computers, set-top boxes, personal data assistants, electronic book readers and the like. Examples of such recipients or users may include medical providers including therapists, or patients. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes a Web server 506 for receiving requests and serving content in response thereto, although for other networks, an alternative device serving a similar purpose could be used, as would be apparent to one of ordinary skill in the art.

The illustrative environment includes at least one application server 508 and a data store 510. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server 508 can include any appropriate hardware and software for integrating with the data store 510 as needed to execute aspects of one or more applications for the client device and handling a majority of the data access and business logic for an application. The application server provides access control services in cooperation with the data store and is able to generate content such as text, graphics, audio and/or video to be transferred to the user, which may be served to the user by the Web server 506 in the form of HTML, XML or another appropriate structured language in this example. The handling of all requests and responses, as well as the delivery of content between the client device 502 and the application server 508, can be handled by the Web server 506. It should be understood that the Web and application servers are not required and are merely example components, as structured code discussed herein can be executed on any appropriate device or host machine as discussed elsewhere herein.

The data store 510 can include several separate data tables, databases or other data storage mechanisms and media for storing data relating to a particular aspect. For example, the data store illustrated includes mechanisms for storing biomarker data (e.g., production data) 512 and user information 516, which can be used to serve content for the production side. The data store is also shown to include a mechanism for storing log or session data 514. It should be understood that there can be many other aspects that may need to be stored in the data store, such as page image information and access rights information, which can be stored in any of the above listed mechanisms as appropriate or in additional mechanisms in the data store 510. The data store 510 is operable, through logic associated therewith, to receive instructions from the application server 508 and obtain, update or otherwise process data in response thereto. In one example, a user might submit a search request for a certain type of item. In this case, the data store might access the user information to verify the identity of the user and can access the catalog detail information to obtain information about items of that type. The information can then be returned to the user, such as through a patient or therapist portal including biomarker and diagnosis data accessible through a Web page that the user is able to view via a browser on the user device 502. Information for a particular item of interest can be viewed in a dedicated page or window of the browser.

Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 5. Thus, the depiction of the system 500 in FIG. 5 should be taken as being illustrative in nature and not limiting to the scope of the disclosure.

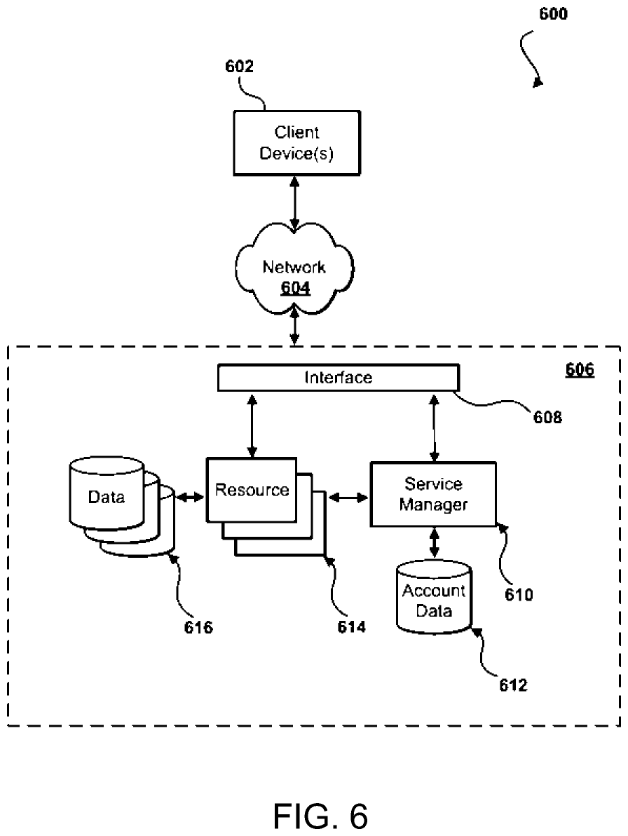

FIG. 6 illustrates an example environment 600 in which aspects of the various embodiments can be implemented. In this example a user is able to utilize a client device 602 to submit requests across at least one network 604 to a multi-tenant resource provider environment 606. The client device can include any appropriate electronic device operable to send and receive requests, messages, or other such information over an appropriate network and convey information back to a user of the device. Examples of such client devices include personal computers, tablet computers, smart phones, notebook computers, and the like. The at least one network 604 can include any appropriate network, including an intranet, the Internet, a cellular network, a local area network (LAN), or any other such network or combination, and communication over the network can be enabled via wired and/or wireless connections. The resource provider environment 606 can include any appropriate components for receiving requests and returning information or performing actions in response to those requests. As an example, the provider environment might include Web servers and/or application servers for receiving and processing requests, then returning data, Web pages, video, audio, or other such content or information in response to the request.

In various embodiments, the provider environment may include various types of resources that can be utilized by multiple users for a variety of different purposes. As used herein, computing and other electronic resources utilized in a network environment can be referred to as “network resources.” These can include, for example, servers, databases, load balancers, routers, and the like, which can perform tasks such as to receive, transmit, and/or process data and/or executable instructions. In at least some embodiments, all or a portion of a given resource or set of resources might be allocated to a particular user or allocated for a particular task, for at least a determined period of time. The sharing of these multi-tenant resources from a provider environment is often referred to as resource sharing, Web services, or “cloud computing,” among other such terms and depending upon the specific environment and/or implementation. In this example the provider environment includes a plurality of resources 614 of one or more types. These types can include, for example, application servers operable to process instructions provided by a user or database servers operable to process data stored in one or more data stores 616 in response to a user request. As known for such purposes, the user can also reserve at least a portion of the data storage in a given data store. Methods for enabling a user to reserve various resources and resource instances are well known in the art, such that detailed description of the entire process, and explanation of all possible components, will not be discussed in detail herein.

In at least some embodiments, a user wanting to utilize a portion of the resources 614 can submit a request that is received to an interface layer 608 of the provider environment 606. The interface layer can include application programming interfaces (APIs) or other exposed interfaces enabling a user to submit requests to the provider environment. The interface layer 608 in this example can also include other components as well, such as at least one Web server, routing components, load balancers, and the like. When a request to provision a resource is received to the interface layer 608, information for the request can be directed to a service manager 610 or other such system, service, or component configured to manage user accounts and information, resource provisioning and usage, and other such aspects. A service manager 610 receiving the request can perform tasks such as to authenticate an identity of the user submitting the request, as well as to determine whether that user has an existing account with the resource provider, where the account data may be stored in at least one account data store 612 in the provider environment. A user can provide any of various types of credentials in order to authenticate an identity of the user to the provider. These credentials can include, for example, a username and password pair, biometric data, a digital signature, or other such information. The provider can validate this information against information stored for the user. If the user has an account with the appropriate permissions, status, etc., the resource manager can determine whether there are adequate resources available to suit the user's request, and if so can provision the resources or otherwise grant access to the corresponding portion of those resources for use by the user for an amount specified by the request. This amount can include, for example, capacity to process a single request or perform a single task, a specified period of time, or a recurring/renewable period, among other such values. If the user does not have a valid account with the provider, the user account does not enable access to the type of resources specified in the request, or another such reason is preventing the user from obtaining access to such resources, a communication can be sent to the user to enable the user to create or modify an account, or change the resources specified in the request, among other such options.

Once the user is authenticated, the account verified, and the resources allocated, the user can utilize the allocated resource(s) for the specified capacity, amount of data transfer, period of time, or other such value. In at least some embodiments, a user might provide a session token or other such credentials with subsequent requests in order to enable those requests to be processed on that user session. The user can receive a resource identifier, specific address, or other such information that can enable the client device 602 to communicate with an allocated resource without having to communicate with the service manager 610, at least until such time as a relevant aspect of the user account changes, the user is no longer granted access to the resource, or another such aspect changes.

The service manager 610 (or another such system or service) in this example can also function as a virtual layer of hardware and software components that handles control functions in addition to management actions, as may include provisioning, scaling, replication, etc. The resource manager can utilize dedicated APIs in the interface layer 608, where each API can be provided to receive requests for at least one specific action to be performed with respect to the data environment, such as to provision, scale, clone, or hibernate an instance. Upon receiving a request to one of the APIs, a Web services portion of the interface layer can parse or otherwise analyze the request to determine the steps or actions needed to act on or process the call. For example, a Web service call might be received that includes a request to create a data repository.

An interface layer 608 in at least one embodiment includes a scalable set of user-facing servers that can provide the various APIs and return the appropriate responses based on the API specifications. The interface layer also can include at least one API service layer that in one embodiment consists of stateless, replicated servers which process the externally-facing user APIs. The interface layer can be responsible for Web service front end features such as authenticating users based on credentials, authorizing the user, throttling user requests to the API servers, validating user input, and marshalling or unmarshalling requests and responses. The API layer also can be responsible for reading and writing database configuration data to/from the administration data store, in response to the API calls. In many embodiments, the Web services layer and/or API service layer will be the only externally visible component, or the only component that is visible to, and accessible by, users of the control service. The servers of the Web services layer can be stateless and scaled horizontally as known in the art. API servers, as well as the persistent data store, can be spread across multiple data centers in a region, for example, such that the servers are resilient to single data center failures.

The various embodiments can be further implemented in a wide variety of operating environments, which in some cases can include one or more user computers or computing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof. In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C# or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc. Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information.

The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed. Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

What is claimed is:

1. A computer-implemented method, comprising:
transcribing, into one or more transcripts, one or more recordings of a session related to an administered therapy for an individual;
parsing the one or more transcripts into utterances;
determining an utterance sentiment for individual utterances; and
predicting an outcome of the individual's response to the administered therapy based, at least in part, upon the utterance sentiment.

2. The computer-implemented method of claim 1, further comprising:
computing sentiment scores for the utterance sentiment, wherein the sentiment scores are indicative of the individual's response to the administered therapy.

3. The computer-implemented method of claim 1, wherein the response to the administered therapy is predicted for treatment-resistant depression.

4. The computer-implemented method of claim 1, further comprising:
generating the one or more recordings;
assigning sentiment scores to individual utterances to determine the utterance sentiment; and
computing session averages of the sentiment scores.

5. The computer-implemented method of claim 4, wherein the sentiment scores include arousal scores and valence scores associated with individual utterances.

6. The computer-implemented method of claim 1, wherein the response to the administered therapy is predicted based, at least in part, upon one or more machine learning models.

7. A computing system, comprising:
a computing device processor; and
a memory device including instructions that, when executed by the computing device processor, enable the computing system to:
transcribe, into one or more transcripts, one or more recordings of a session related to an administered therapy for an individual;
parse the one or more transcripts into utterances;
determine an utterance sentiment for individual utterances; and
using a machine learning model to predict an outcome of the individual's response to the administered therapy based, at least in part, upon the utterance sentiment.

8. The computing system of claim 7, wherein the instructions, when executed by the computing device processor, enable the computing system to further:
determine sentiment scores for the utterance sentiment, wherein the sentiment scores are indicative of the individual's response to the administered therapy.

9. The computing system of claim 7, wherein the individual's response to the administered therapy is predicted for treatment-resistant depression.

10. The computing system of claim 7, wherein the instructions, when executed by the computing device processor, enable the computing system to further:
generate the one or more recordings;
assign sentiment scores to individual utterances to determine the utterance sentiment; and
compute session averages of the sentiment scores.

11. The computing system of claim 10, wherein the sentiment scores include arousal scores and valence scores associated with individual utterances.

12. The computing system of claim 7, wherein the response to the administered therapy is predicted based, at least in part, upon one or more machine learning models.

13. The computing system of claim 7, wherein the utterance sentiment is determined utilizing a classifier built on a large language model.

14. A non-transitory computer-readable medium storing instructions that, when executed by at least one processor, cause the at least one processor to:
transcribe, into one or more transcripts, one or more recordings of a session related to an administered therapy for an individual;
parse the one or more transcripts into utterances;
determine an utterance sentiment for individual utterances; and
predict the individual's response to the administered therapy based, at least in part, upon the utterance sentiment.

15. The non-transitory computer-readable medium of claim 14, wherein the instructions, when executed by the at least one processor, cause the at least one processor to further:
determine sentiment scores for the utterance sentiment, wherein the sentiment scores are indicative of the individual's response to the administered therapy.

16. The non-transitory computer-readable medium of claim 14, wherein the individual's response to the administered therapy is predicted for treatment-resistant depression.

17. The non-transitory computer-readable medium of claim 14, wherein the instructions, when executed by the at least one processor, cause the at least one processor to further:
generate the one or more recordings;
assign sentiment scores to individual utterances to determine the utterance sentiment; and
compute session averages of the sentiment scores.

18. The non-transitory computer-readable medium of claim 17, wherein the sentiment scores include arousal scores and valence scores associated with individual utterances.

19. The non-transitory computer-readable medium of claim 14, wherein the response to the administered therapy is predicted based, at least in part, upon one or more machine learning models.

20. The non-transitory computer-readable medium of claim 14, wherein the utterance sentiment is determined utilizing a classifier built on a large language model.
